(19) United States
(12) Patent Application Publication
(10) Pub. No.: US 2025/0389953 A1
(43) Pub. Date: Dec. 25, 2025

(54) METHODS AND SYSTEMS FOR A SIMULATED FIELD OF VIEW AFFECTED BY AN OPHTHALMIC CONDITION

(71) Applicant: Lenz Therapeutics Operations, Inc., Del Mar, CA (US)

(72) Inventors: Shawn OLSSON, Long Grove, IL (US); Marc Geoffrey ODRICH, Charlottesville, VA (US); Gerald HORN, Highland Park, IL (US)

(21) Appl. No.: 18/753,403

(22) Filed: Jun. 25, 2024

(51) Int. Cl. G02B 27/00

(52) U.S. Cl. CPC G02B 27/0025; G02B 27/0075

(57) ABSTRACT

Provided herein are methods and computer-implemented systems for simulating a field of view affected by an ophthalmological condition. The ophthalmological condition may be presbyopia. The method may comprise receiving from a user interface one or more parameters relating to said ophthalmic condition. The method may comprise receiving, from a head mounted display, sensor data and a video stream. The method may comprise using one or more computer processors, applying an algorithm to modify at least a portion of said video stream to generate a simulated field of view, wherein said modification is based at least in part on said sensor data, said video stream, and said one or more parameters. The method may comprise providing said simulated field of view affected by an ophthalmic condition to a user via a graphical interface. The sensor data may include location data of one or more objects within the field of view. The algorithm may be applied to the object based on its location from the head mounted display.

### BACKGROUND

The National Institutes of Health estimates that nearly 80% of people in North America have presbyopia by age 55. Other ophthalmic conditions, such as cataracts, childhood myopia, astigmatism, macular degeneration, and glaucoma affect subject's field of view in ways that are difficult to show to non-subjects.

### SUMMARY

Existing representations of ophthalmic conditions are difficult to adapt to individual user's experiences. Accurate estimations and representations of levels of severity of ophthalmic conditions are critical to patient decisions regarding treatment options, resources allocated to treatment research, and comparison of treatment options. There exists a need for tools that enable individuals to experience accurate simulations of ophthalmic conditions. There also exists a need for those simulations to be tailored to the individual's environment and be tailored to a specific type and severity of ophthalmological condition.

Furthermore, when applying AR and VR technologies to ophthalmic conditions, such as presbyopia, there exists a problem of accurate alteration of a simulated ophthalmic condition. As described herein, a discovered solution to provide more accurate simulation involves the measurement of distance of objects from a point of origin of the field of view. This enables more accurate image classification, more precise application of image alteration algorithms, and more realistic experiences provided to a user.

The systems and methods described herein provide for real-time simulations of ophthalmological conditions. These systems and methods may be used as tools to give an individual without an ophthalmological condition an improved understanding of the ophthalmological condition. Treatment providers and decision makers may be better equipped to evaluate ophthalmological conditions and their treatment options by switching between multiple simulated experiences in real time. Individuals may better understand the impacts of an ophthalmological condition on specific aspects of an affected individual's life by simulating an ophthalmological condition when viewing their real-world surroundings.

In one aspect, provided herein is a computer-implemented system for simulating an ophthalmic condition comprising. The system comprises a head mounted display and a computing device comprising at least one processor, an operating system configured to perform executable instructions, a memory, and a computer program including instructions executable by the at least one processor to cause the at least one processor to perform operations comprising: receiving from a user interface a type of the ophthalmic condition and one or more parameters relating to the type of the ophthalmic condition; receiving, from the head mounted display, at least sensor data and video; applying an algorithm to alter the video based on at least the sensor data, the video, and the one or more parameters relating to the type of ophthalmic condition; and providing a simulation of the ophthalmic condition to a user via the head mounted display.

In some cases, the type of the ophthalmological condition is presbyopia. In some cases, the type of ophthalmological condition is presbyopia. In some cases, the video stream is of an environment of the user. In some cases, the algorithm is applied to the video stream in real time. In some cases, the head mounted display determines a distance of a first object in the field of view. In some cases, the head mounted display comprises an accelerometer. In some cases, the head mounted display comprises a gyroscope. In some cases, the algorithm locates the first object in the field of view, determines a distance of the first object from the head mounted display, and applies the algorithm based on the distance of the first object to the head mounted display. In some cases, the algorithm locates a second object in the field of view, determines a distance of the second object from the head mounted display, and applies the algorithm based on the distance of the second object to the head mounted display. In some cases, the algorithm segments the field of view into a plurality of objects, determines a distance of each object of at least a subset of the plurality of objects based on the sensor data received from the head mounted display, and applied the algorithm to the video of the plurality of objects based on the distance of each object. In some cases, the computer-implemented method further comprises providing an output. In some cases, the output comprises a summary of the quantity of objects which the algorithm modified. In some cases, the output comprises a summary of the magnitude of the modification of one or more objects. In some cases, the output comprises a recording of the simulated field of view affected by the ophthalmic condition. In some cases, the operations further comprise one or more of the following: splitting the field of view into one or more image segments; identifying one or more objects within the field of view; classifying one or more objects based on or more factors; and applying a distortion filter. In some cases, the algorithm comprises classifying one or more objects based on at least the one or more object's distance from the head mounted display. In some cases, the one or more parameters further comprise an age of the subject. In some cases, the field of view is portioned into at least two sections and where only a first section is modified. In some cases, the field of view is collected and modified in real-time. In some cases, the modifying the at least a portion of the field of view comprises blurring components of the field of view. In some cases, the components are blurred based on their distance from origin of the field of view. In some cases, the one or more parameters comprises a severity of the ophthalmic condition. In some cases, the ophthalmic condition is cataracts. In some cases, the ophthalmic condition is myopia. In some cases, the one or more parameters comprises refractive error. In some cases, the operations further comprise providing a second modification of the field of view based at least in part on a simulated treatment parameter. In some cases, the field of view is generated by a graphical interface of a smartphone. In some cases, the applying an algorithm to alter the video comprises blurring text located less than or equal to a provided distance from the point of origin of the field of view. In some cases, the provided distance is based on a simulated or real age of the subject. In some cases, at least a portion of the field of view is not altered.

In one aspect, provided herein is a method of simulating a field of view affected by an ophthalmic condition, comprising: receiving from a user interface one or more parameters relating to the ophthalmic condition; receiving, from a head mounted display, sensor data and a video stream; using one or more computer processors, applying an algorithm to modify at least a portion of the video stream to generate a simulated field of view, wherein the modification is based at least in part on the sensor data, the video stream, and the one or more parameters; and providing the simulated field of view affected by an ophthalmic condition to a user via a graphical interface.

In some cases, the method further comprises, running a diagnostic on the subject to determine the ophthalmic condition or the one or more parameters relating to the ophthalmic condition. In some cases, the method further comprises, providing the ophthalmic condition or the parameter relating to the ophthalmic condition to the one or more computer processors. In some cases, the type of the ophthalmological condition is presbyopia. In some cases, the video stream is of an environment of the user. In some cases, the algorithm is applied to the video stream in real time. In some cases, the head mounted display determines a distance of a first object in the field of view. In some cases, the head mounted display comprises an accelerometer. In some cases, the head mounted display comprises a gyroscope. In some cases, the algorithm locates the first object in the field of view, determines a distance of the first object from the head mounted display, and applies the algorithm based on the distance of the first object to the head mounted display. In some cases, the algorithm locates a second object in the field of view, determines a distance of the second object from the head mounted display, and applies the algorithm based on the distance of the second object to the head mounted display. In some cases, the algorithm segments the field of view into a plurality of objects, determines a distance of each object of at least a subset of the plurality of objects based on the sensor data received from the head mounted display, and applied the algorithm to the video of the plurality of objects based on the distance of each object. In some cases, the method further comprises providing an output. In some cases, the output comprises a summary of the quantity of objects which the algorithm modified. In some cases, the output comprises a summary of the magnitude of the modification of one or more objects. In some cases, the output comprises a recording of the simulated field of view affected by the ophthalmic condition. In some cases, the method further comprises one or more of the following: splitting the field of view into one or more image segments; identifying one or more objects within the field of view; classifying one or more objects based on or more factors; and applying a distortion filter. In some cases, the algorithm comprises classifying one or more objects based on at least the one or more object's distance from the head mounted display. In some cases, the one or more parameters further comprise an age of the subject. In some cases, the field of view is portioned into at least two sections and where only a first section is modified. In some cases, the field of view is collected and modified in real-time. In some cases, the modifying the at least a portion of the field of view comprises blurring components of the field of view. In some cases, the components are blurred based on their distance from origin of the field of view. In some cases, the one or more parameters comprises a severity of the ophthalmic condition. In some cases, the ophthalmic condition is cataracts. In some cases, the ophthalmic condition is myopia. In some cases, the one or more parameters comprises refractive error. In some cases, the operations further comprise (e) providing a second modification of the field of view based at least in part on a simulated treatment parameter. In some cases, the applying an algorithm to alter the video comprises blurring text located less than or equal to a provided distance from the point of origin of the field of view. In some cases, the provided distance is based on a simulated or real age of the subject. In some cases, the provided distance is adjusted dynamically in real-time. In some cases, at least a portion of the field of view is not altered.

Non-transitory computer-readable storage media encoded with a computer program including instructions executable by one or more processors to create a simulated field of view comprising: a database, in a computer memory, of a video of a field of view, outputs from one or more sensors from a head mounted device, one or more parameters collected from a graphical user interface; a software module receiving video and sensor data from an HMD; and a software module applying an algorithm to alter the video based at least in part of the outputs of the one or more sensors from the head mounted display and return the altered video to the head mounted display.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a computing device; in this case, a device with one or more processors, memory, storage, and a network interface.

FIG. 2 shows a non-limiting example of a web/mobile application provision system; in this case, a system providing browser-based and/or native mobile user interfaces.

FIG. 3 shows a non-limiting example of a cloud-based web/mobile application provision system; in this case, a system comprising an elastically load balanced, auto-scaling web server and application server resources as well synchronously replicated databases.

FIG. 4 shows a non-limiting example of a flowchart outlining a method of providing an altered field of view to a user, in accordance with some embodiments described herein.

FIG. 5 shows a non-limiting example of a flowchart outlining a method of providing a simulated field of view to a user, in accordance with some embodiments described herein.

### DETAILED DESCRIPTION

As used herein, the terms “head mounted display” and “head mounted device” may refer to each other, or other types of wearable devices that enable display of video and audio experiences to a user. Head mounted display may be or comprise a smartphone positioned adjacent to the head of the user.

As used herein, a type of an ophthalmic condition may refer to a diagnosis, an indication, or a description of an ophthalmic condition. A type of ophthalmic condition may also refer to a level of severity of an ophthalmic condition. For example, high presbyopia may be a type of ophthalmic condition.

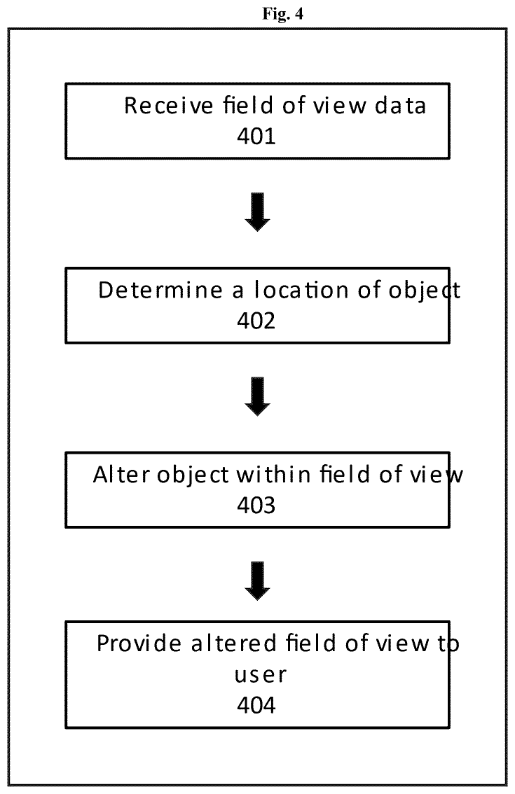

FIG. 4 shows an example of a process to provide an altered field of view to a user. First, data of a field of view is received by one or more computer processors 401. The one or more processors determine a location of an object in the field of view 402. The one or more computer processors apply an algorithm to alter the object within the field of view 403. The one or more processors provide the altered field of view to a user via a head mounted display 404.

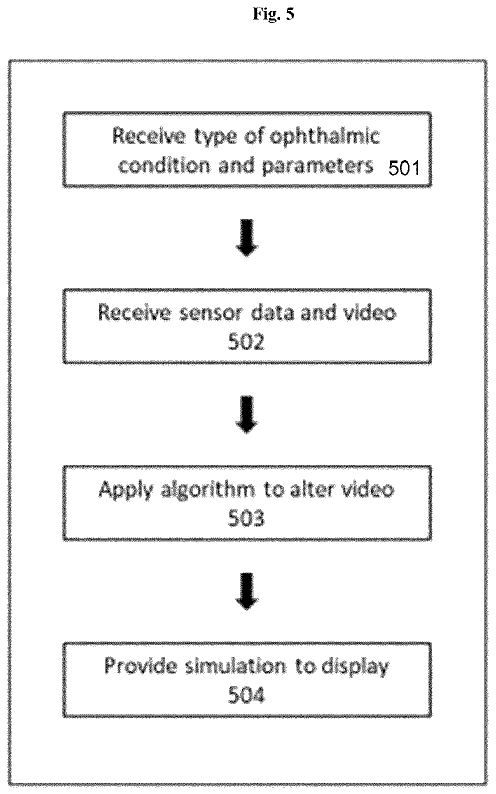

FIG. 5 shows an example of a process to provide a simulated field of view to a head mounted display, as described further herein. The process comprises receiving a type of ophthalmic condition and one or more parameters related to the ophthalmological condition 501. The process comprises receiving sensor data and video stream from a head mounted display of the field of view 502. The head mounted display applies an algorithm to alter the video feed based at least in part on the sensor data 503. The head mounted display provides a simulated field of view to the user 504.

### Partial Field of View

In certain embodiments, the field of view provided to the user (e.g., provided on a graphical interface of the head mounted display) is portioned into at least two parts. A first part may have a different magnitude or type of image alteration applied by the algorithm to generate that part of the simulated field of view (e.g., video or augmented reality). For example, the field of view may be partitioned into two sections. The two sections may be about half of the field of view. The sections may be a left half and a right half. The section may be a top half and a bottom half. The algorithm may alter the field of view (e.g., altered video or augmented reality) so that a first half simulates an ophthalmological condition. Simultaneously, the second half of the field of view shows an unaltered field of view. Simulating two portions of a field of view, a first with a simulated ophthalmological condition and the second without, would allow a user to compare the quality of each portion of the field of view against the other, or another portion of the field of view.

The simulated field of view may be partitioned into a plurality of portions. The section of the field of view in each partition may be simulated to a magnitude or type of ophthalmological condition that is the same or different from one or more of the other portions of the plurality of portions of the simulated field of view.

In some cases, the partitioned field of view may be labeled to indicate a severity, type, or other parameter relating to an ophthalmological condition. For example, the field of view may be partitioned into two sections, where a first section simulates a field of view affected by presbyopia. The first section may be labeled to indicate that section is altered to simulate presbyopia. In some cases, the section may be labeled to indicate a severity of the simulated ophthalmological condition. For example, a first section may be labeled by category of presbyopia (e.g., mild presbyope, moderate presbyope, or advanced presbyope) of the simulated field of view. A benefit of providing two or more partitions (i.e., sections) of the simulated field of view is to allow comparison between different simulations in real time. A user can change a perspective of the field of view. The user may adjust the orientation of the head mounted display. Alternatively, or in addition, a video provided to the graphical interface to generate the simulated field of view may represent an environment (i.e., one or more objects) moving through one or more sections of the field of view. This may allow a user to experience a constant object move through one or more section to compare the effects of the simulated condition of that section on the object.

### Real-Time

The term “in real-time” and “real-time,” as used herein, generally refers to immediate, rapid, not requiring operator intervention, automatic, and/or programmed. Real-time may include, but is not limited to, measurements in femtoseconds, picoseconds, nanoseconds, milliseconds, seconds, as well as longer, and optionally shorter, time intervals. In the systems and methods described herein, one or more steps of a process (e.g., a method or a step of a computer implemented system's code) may be performed in real-time. In some cases, a simulated field of view may be provided in real-time. The simulated field of view may be adjusted in real time. The simulated field of view may be controlled by the user, or a non-user in real time.

### Augmented Reality vs Virtual Reality

As used herein, “augmented reality” may refer generally to a field of view that comprises at least a portion of a true or real-world field of view. For example, a person physically located in a dark room would perceive, in at least a portion of their field of view, at least a portion of the dark room.

Augmented reality simulated field of view may comprise a pass through field of view. Alternatively, or in addition, an augmented reality field of view may comprise a graphical interface generated field of view. The pass through field of view would provide at least a portion of the signals on the visible light spectrum from an environment of a user directly to an eye of the user. In contrast, a field of view generated by a graphical interface would generate and transmit signals on the visible light spectrum to the eye of a user. For augmented reality, a graphical user interface would generate and transmit signals that represent, or are based on, the surroundings of the user.

As used herein, “virtual reality” may refer generally to a field of view that does not comprise a true or real-world field of view of the environments of the user experiencing the virtual reality experience. For example, a user in a dark room does not perceive any portion of the dark room, but instead views a complete field of view provided on a graphical user interface to the user. Virtual reality must be a graphical interface generated field of view.

### Pass Through and Graphical Interface Generated Fields of View

As used herein, “pass through” viewing may refer to a field of view where at least a portion of the signals on the visible light spectrum of the environment surrounding a user reach the eye of the user. For example, a user looking through a clear, or at least partially-clear component would view at least a portion of true or real-world field of view.

In some cases, a simulated field of view may comprise at least a part of a pass through field of view. Alternatively, or in addition, the simulated field of view may generate a filter or alteration to the visible light signals passing through an element in the field of view. The filter or element may alter the field of view to generate the simulated field of view. A pass through field of view may comprise a graphical interface that is at least partially clear or non-opaque.

In contrast to a pass through simulated field of view, a field of view may be entirely generated by a graphical interface. The entirety of the signals on the visible light spectrum reaching an eye of the user are generated by the graphical user interface of the device providing the virtual reality experience.

### Image Alterations

In some cases, an image alteration in a simulated field of view described herein may comprise a blur. The blur may comprise one or more pixels (e.g., an object or section of the field of view) that are adjusted to reflect a local average value. For example, 9 pixels in a 3 by 3 grid may be blurred by adjusting all 9 pixels to the average value of the 9 pixels. Each pixel of the blur alteration may be based on an average value of surrounding pixels, and may be inclusive of the original pixel value.

In some cases, an image alteration in a simulated field of view described herein may comprise an astigmatism. For example, an algorithm described herein may identify pixels (e.g., objects or locations withing the field of view) that have brighter pixel values than other pixels (e.g., surrounding pixels, objects, sections, or an average value for the field of view. The algorithm may extend the bright pixel value along one or two linear directions to simulate the effect of an astigmatism.

In some cases, an image alteration in a simulated field of view described herein may comprise an opaque alteration. The algorithm may reduce a brightness or contrast quality of one or more pixels (e.g., an object or section of the field of view) to create a partially or completely opaque filter.

In some cases, the simulated field of view may comprise one or more pixels (e.g., an object or section of the field of view) that are completely obstructed. This may simulate a cataract or other ophthalmic condition that restricts a field of view.

In some cases, the simulated field of view may comprise brightness adjustment. Brightness adjustment may simulate an ophthalmic condition affecting the cornea or pupil of an affected eye.

### Dynamic Alterations

In some cases, the alterations to the field of view (e.g., video feed or pass through field of view) may be adjusted in real time to create the simulated field of view that is dynamically changed. The simulated field of view may be adjusted by one or more inputs from a user interface. The one or more inputs may be provided by a user experiencing the simulated field of view. Alternatively, or in addition, the inputs may be provided by an individual who is not experiences the simulated field of view (e.g., is not a user of the head mounted display). For example, a user wearing the head mounted display is experiencing an augmented reality simulated field of view of their surroundings. A second individual, not wearing the head mounted display, provides an input to the computing device to adjust the alterations to the field of view (e.g., the pass through field of view or graphically provided field of view). The input may provide instructions to the computing device to adjust a severity of the ophthalmological condition of the simulated field of view. The input may adjust a field of view to simulate a changing severity of the ophthalmological condition (e.g., as the simulated subject ages).

### Types of Parameters for Ophthalmological Conditions

The systems and methods described herein may simulated an ophthalmological condition. The ophthalmological condition may be presbyopia, myopia, cataracts, astigmatism, macular degeneration, visual field loss due to glaucoma, hemianopsia secondary to cerebral vascular accident or stroke. For an ophthalmological condition, one or more parameters relating to the ophthalmological condition may be provided to the computing device of the system described herein. The one or more parameters may include, but are not limited to, age of a simulated user, severity of presbyopia, severity of myopia, location of one or more cataracts, severity of one or more cataracts, extent of partial vision loss due to hemianopsia, vision loss due to glaucoma. In some cases, the parameters may describe a two-dimensional schematic of partial vision loss or affected vision.

In some cases, the one or more parameters are derived from a diagnosis of a subject. The simulated field of view may represent the subject's ophthalmologic diagnosis. The simulated field of view may allow a user (e.g., not the subject) to experience a representation of the vision experience of the subject. The ophthalmological diagnosis may comprise a vision score, a classification of presbyope type, a type of cataracts, or other ophthalmological condition diagnosis and parameters relating to that ophthalmological condition. The one or more parameters may relate to, or be a severity of an ophthalmic condition.

In some cases, the one or more parameters relate to a treatment option for an ophthalmological condition. The simulated field of view may provide a representation of an expected field of view during or after treatment. The simulated field of view may dynamically change between one or more treatment options. The simulated field of view may dynamically change between one or more treatment options and a non-treatment state. The non-treatment state may be a current type and severity of an ophthalmological condition. The non-treatment state may be a projected type and severity of an ophthalmological condition.

### Distance Measurement

In some cases, an ophthalmological condition simulated through augmented reality or virtual reality by a system or method described herein may be based at least in part on a distance of one or more objects in the simulated field of view.

For augmented reality, where the simulated field of view is based on the real-world surroundings of a user, the head mounted display may comprise one or more sensors. The one or more sensors may comprise distance sensors (e.g., sensors configured to measure a distance of one or more objects from the head mounted display). The one or more sensors may comprise an accelerometer, a gyroscope, or other positioning sensors.

The one or more sensors may measure a distance between an object (e.g., an object in the field of view) and the point of origin of the field of view (e.g., the head mounted display). The sensors may comprise cameras. In some cases, two or more cameras capture two or more video feeds of the environment of user. A computing device may apply an algorithm to compare the two or more video feeds. The computing device may identify one or more objects in the field of view of the video feeds. The computing device may determine a distance from the user of at least a subset of the one or more objects in the field of view.

A camera of the one or more sensors may record a focal setting (e.g., a focal length or location of a focal point). The computing device may calculate a distance of an object in the video stream captured by the camera based on the focal setting of the camera.

### Object Identification

In some cases, an algorithm is applied to one or more objects identified in a field of view to provide the simulated field of view of the ophthalmological condition. The computing device may apply the algorithm to a video feed to identify one or more objects. The algorithm may compare the one or more identified objects to a database of known objects. The algorithm may classify the one or more objects based on the results of the comparison. The algorithm may alter the portion of the field of view (e.g., either by altering the simulated video provided to the user via the graphical interface, or by altering the field of view comprising pass through signals from the environment) based on the classification of the one or more objects in the field of view. As an example, the algorithm may identify an object (e.g., a can of soda) in the field of view. The algorithm may compare the object video to a database of objects (e.g., a database with a set of values relating to a can of soda). The algorithm may classify the object (e.g., as a can of soda). The algorithm may apply a set of alterations to the field of view based on the object's classification (e.g., the algorithm may blur the can of soda in the field of view).

In some cases, an algorithm is applied to identify a pre-programmed object within the field of view. For example, a system for simulating presbyopia, as described herein, may comprise simulating a user reading the nutrition label of a can of soda. The algorithm may identify a blank can (e.g., an object of the same size as a can of soda). The computing device may apply a pre-programmed alteration to create a simulated field of view. The simulated field of view may comprise a blurred soda can label in the place of the recognized object (e.g., the blank can). The one or more computer processors may add a virtual object over the location of a recognized object within the field of view.

The algorithm may alter the field of view based on the classification of the object and one or more parameters indicative of the location of the object from the origin of the field of view (e.g., the user, the head mounted display, the camera recording the video, etc.). The parameters indicative of the location of the object may comprise a size of the object, identification of text on the object, identification of features with a size that may only be recognized if the object is located closer than a threshold distance from the origin of the field of view. For example, the algorithm may identify the can of soda in the field of view. The algorithm may identify text on the can of soda (e.g., in the nutrition label, on the wrapper of the can, etc.). The algorithm may determine a distance of the can of soda based on the size of the identified text relative to the field of view. Alternatively, or in addition, the algorithm may determine a distance of the object (e.g., the can of soda) based on the size of the object (e.g., can of soda) relative to the field of view, and a comparison to a known value of the size of the object. For example, the algorithm may determine the size of the can of soda relative to the field of view. If the relative size of the can of soda is greater than a stored threshold value, the algorithm may classify the can of soda as being closer than a corresponding threshold distance (e.g. a distance classification). The algorithm may apply an alteration of the image of the object in the simulated field of view based on the distance classification of the can of soda.

### Outputs of Simulation

In some embodiments, the methods and computer-implemented systems described herein may generate an output. The output may describe or relate to the simulated field provided by the head mounted display to the user. For example, the output may comprise a score of the affected vision simulated by the head mounted display. The output may comprise a score by the user on the difficulty of performing a task with the simulated vision. The output may comprise a qualitative or quantitative metric to describe the simulated field of view or the user's experience of the simulated field of view. In some cases, the output may be provided to a health care professional. The output may be used in determining a treatment plan for the user. For example, a user scoring a simulated field of view (e.g., presbyopia) below a certain threshold may be used to recommend a treatment for presbyopia to the user.

In some cases, an output may comprise a recording of the user's simulated field of view. The recording may be provided to the user at a later time point. The recording may be provided to a third party (e.g., not the user). For example, the recording of a child's field of view may be provided to a decision maker for that child's health care (e.g., a parent or guardian of the child) to be used in determining a treatment plan for the child. In some cases, the video feed provided to the head mounted display may be a recording of a first person's field of view for a period of time, then altered to simulate an ophthalmic condition of the user (i.e., currently or modeled) and provided to a second person. The first person may be a child with a progressing ophthalmic condition (e.g., myopia) and the second person may be a decision maker for that child (e.g., a parent or guardian). This may be useful as a tool to show a health decision maker an extent to which an ophthalmic condition of another person affects that other person. This solution meets a need for tools to enable decision makers better insight into the condition of other person's ophthalmic condition. For example, a parent may not realize the extent that an ophthalmic condition affects their child's vision. Additionally, the parent may not realize how many objects are within that affected field of vision for the child.

In some cases, an output may comprise a score or description of a quality of life, speed to complete a task, or other metric compared against a similar experience without the simulated ophthalmic condition. For example, a person is given a task involving reading and writing text. The person is scored on completing the task (e.g., timing, accuracy and quality of completion) without an affected field of vision. Then the person is scored on completing the same task with a simulated ophthalmic condition affecting their field of vision. The output may comprise a value comparing the difference between the scores of the affected and non-affected tasks.

### Certain Definitions

Unless otherwise defined, all technical terms used herein have the same meaning as commonly understood by one of ordinary skill in the art to which the present subject matter belongs.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

### Computing System

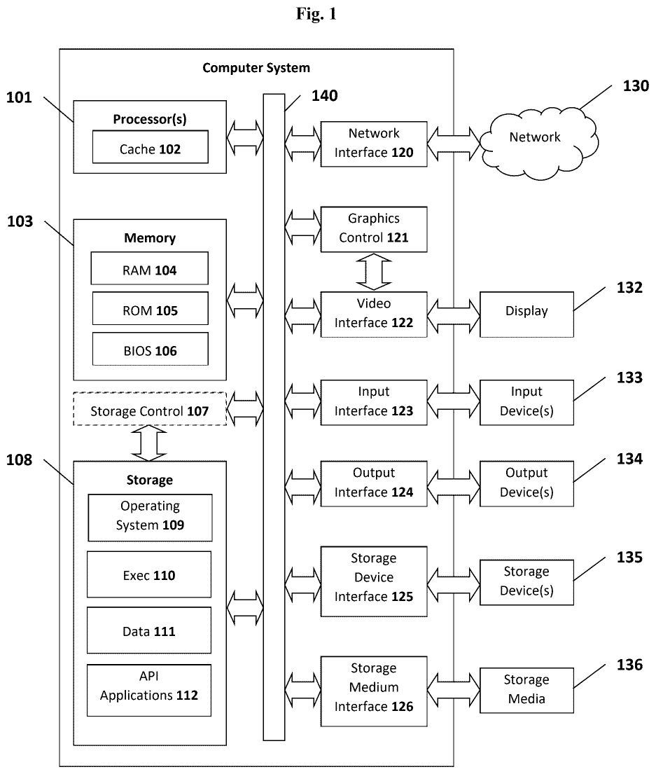

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition, or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, cloud computing platforms, distributed computing platforms, server clusters, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS® Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

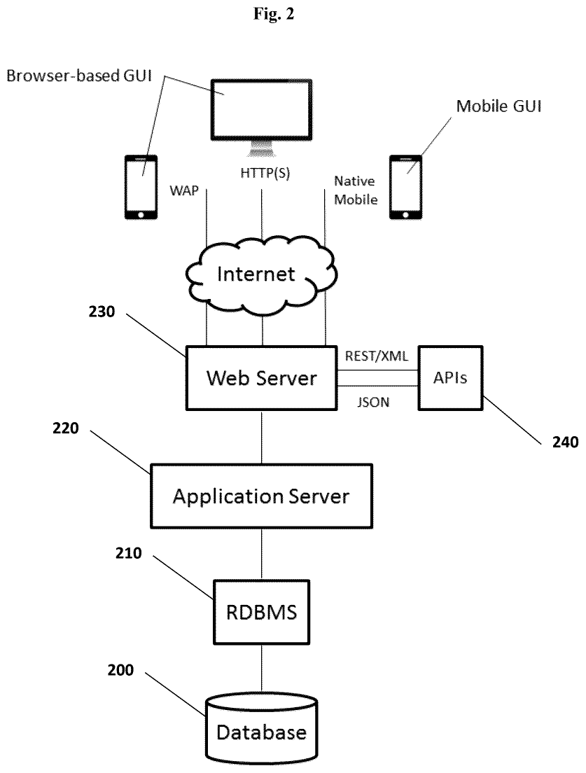

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

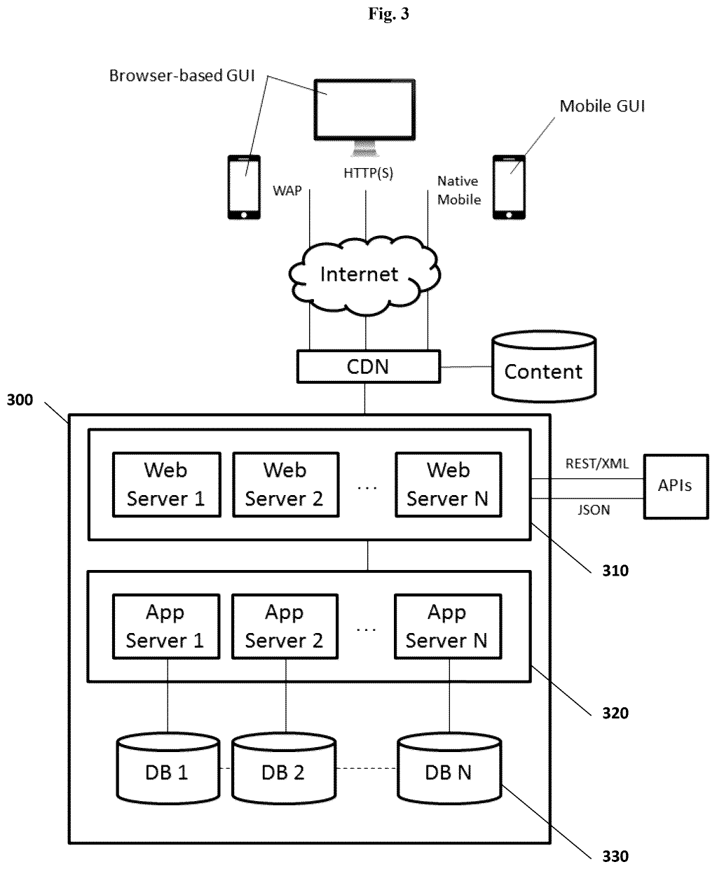

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of virtual reality information, including but not limited to video, audio and location information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### EXAMPLES

The following illustrative examples are representative of embodiments of the software applications, systems, and methods described herein and are not meant to be limiting in any way.

### Example 1—Local Virtual Reality Simulation of Presbyopia

In one non-limiting example, a computer-implemented system described herein simulates presbyopia, an ophthalmic condition. The system comprises a head mounted display and a computing device. The computing device may be in the head mounted display, or located remotely from the head mounted display. The computing device receives, through a user interface, that the ophthalmic condition is presbyopia. The computing device receives parameters relating to presbyopia, including but not limited to, an amount of blur caused by presbyopia, a distance from a user's point of view. A parameter may be a set of distances, or a range of distances, to indicate near vision, intermediate vision, and distance vision. Near vision may be defined by the distance range from a point of origin of a field of view of less than or equal to about 40 centimeters. Intermediate vision may be defined by the distance range from the point of origin of the field of view of between about 40 cm and about 100 cm. Distance vision may be defined by distances from the point of origin of the field of view of greater than or equal to about 100 centimeters. The head mounted display may capture video of the surroundings of the head mounted display. The head mounted display may use one or more sensors to collect data on the location of objects in the field of view of the captured video. The video may be captured in real time. The computing device, as described herein, may apply an algorithm to alter the video based on the sensor data and the parameters. The algorithm may blur objects that are determined to be within the near vision range from the origin of the field of view of the video (e.g., the head mounted display). The algorithm may additionally blur objects determined to be in the intermediate vision distance range from the origin of the field of view of the video (e.g., the head mounted display). The blur applied to objects in the intermediate vision distance range may be different from the blur applied to objects in the near vision range. The altered (e.g., blurred) video feed is provided to a user via a graphical interface of the head mounted display. The altered video feed is provided in real time, or near real time. The altered video feed simulates the point of view of the user with presbyopia.

### Example 2—Pre-Loaded Virtual Reality Simulation of Presbyopia

As a non-limiting example, systems and methods described herein may provide a pre-loaded virtual reality simulation of an ophthalmological condition (e.g., presbyopia). A head mounted display may receive an information package describing a three-dimensional environment. The information package may include data relating to the focal point of one or more objects in the three dimensional environment. Using the sensors of the head mounted display and the information package, the computing device may provide a virtual reality experience to the user. The computing device may alter the video feed provided to a graphical interface of the head mounted display to simulate a field of view affected by presbyopia. The computing device may blur objects based on the determined distance from the point of view of the user in the virtual reality experience.

### Example 3—Augmented Reality Simulation of Presbyopia

Systems and methods described herein may provide an augmented reality simulation of presbyopia. The simulated field of view comprises a portion of the real-world or true environment of the user. The system comprising a computing device applies an algorithm to alter the real-world or true environment received through a head mounted device. The head mounted device comprises a viewing element that is at least partially clear. The viewing element may alter the visible light signals passing through the element to an eye of the user. The computing device controls the alteration of the visible light signals based on the algorithm applied to the visible light signals. The algorithm alters the visible light signals passing through the viewing element by sensing the visible light signals as they pass through the viewing element. The head mounted device transmits additional light signals from the viewing element to the user. Alternatively, or in addition, the head mounted device partially or completely blocks a portion of the signals passing through the viewing element. In some cases, the additional signals provided by the viewing element block the signals from the real-world environment.

### Example 4—Example Use Case for Simulated Vision

As a non-limiting example, the systems and methods described herein may provide a simulated field of view to a user. The simulated field of view may simulate an ophthalmic condition (e.g., presbyopia). This may be helpful to allowing a user to experience a scenario where a person with an ophthalmic condition may be affected differently than a person without the ophthalmic condition. As an example, a user is provided a head mounted display comprising a computing device. The user, wearing the head mounted display, is asked to perform a task. For example, the user is asked to respond to a credit card fraud alert. To respond, the user, is asked to locate their credit card and read off the numbers while wearing the head mounted display. The head mounted display simulates a field of view with presbyopia by altering (e.g., blurring) text and other objects located within 40 centimeters from the head mounted display. After experiencing the simulated field of view, the user is prompted to record a characterization of the experience. The characterization may include the severity of the field of view, the difficulty completing the task, the additional time to complete the task, and other qualities of the user's experience. The user may be asked to repeat the task with a different level of severity of ophthalmic condition. For example, the user repeats the task with a high level of presbyopia, wherein the head mounted display blurs text and objects within 90 centimeters from the head mounted display.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

1.-64. (canceled)

65. A computer-implemented system for simulating an ophthalmic condition comprising a head-mounted display comprising a set of sensors, a computing device comprising one or more processors, a memory, and a computer program including instructions that, when executed by the one or more processors, individually or collectively cause the one or more processors to perform operations comprising:
a) receiving, via a user interface, a first input comprising the ophthalmic condition, one or more parameters relating to the ophthalmic condition, or a combination thereof;
b) acquiring, using the set of sensors, real-time sensor data;
c) receiving, from the head-mounted display, a second input comprising the real-time sensor data;
d) computer processing the real-time sensor data to automatically alter at least a portion of the real-time sensor data, based at least in part on the first input, wherein the altering comprises generating simulated effects of the ophthalmic condition; and
e) providing to a user, via the user interface, an electronic output comprising the altered real-time sensor data,
wherein the ophthalmic condition is selected from the group consisting of presbyopia, myopia, cataracts, astigmatism, macular degeneration, visual field loss due to glaucoma, and hemianopsia secondary to cerebral vascular accident or stroke.

66. The computer-implemented system of claim 65, wherein the ophthalmological condition is presbyopia.

67. The computer-implemented system of claim 65, wherein the set of sensors comprises an accelerometer and/or a gyroscope.

68. The computer-implemented system of claim 65, wherein the operations further comprise one or more of:
a. identifying one or more objects within a field of view of the set of sensors;
b. classifying one or more objects based at least in part on or more factors; and
c. applying a distortion filter.

69. The computer-implemented system of claim 65, wherein the one or more parameters further comprise an age of a subject.

70. The computer-implemented system of claim 65, wherein the operations further comprise blurring components of a field of view of the set of sensors, based at least in part on a distance of the components from an origin of the field of view.

71. The computer-implemented system of claim 65, wherein the one or more parameters comprise a severity of the ophthalmic condition.

72. The computer-implemented system of claim 65, wherein the altering further comprises blurring text located less than or equal to a provided distance from a point of origin of a field of view of the set of sensors.

73. A method of simulating a field of view affected by an ophthalmic condition, comprising:
a) receiving, via a user interface, a first input comprising the ophthalmic condition, one or more parameters relating to the ophthalmic condition, or a combination thereof;
b) acquiring, using a set of sensors of a head-mounted display, real-time sensor data comprising a video stream;
c) receiving, from the head-mounted display, the real-time sensor data;
d) computer processing the real-time sensor data to automatically alter at least a portion of the video stream, wherein the altering comprises generating a simulated field of view affected by the ophthalmic condition, based at least in part on the real-time sensor data and the one or more parameters; and
e) providing to a user, via a graphical interface, an electronic output comprising the simulated field of view affected by the ophthalmic condition, wherein the ophthalmic condition is selected from the group consisting of presbyopia, myopia, cataracts, astigmatism, macular degeneration, visual field loss due to glaucoma, and hemianopsia secondary to cerebral vascular accident or stroke.

74. The method of claim 73, wherein the ophthalmological condition is presbyopia.

75. The method of claim 73, wherein d) is performed in real time.

76. The method of claim 73, wherein the set of sensors comprises an accelerometer and/or a gyroscope.

77. The method of claim 73, further comprising performing one or more of:
a. identifying one or more objects within the simulated field of view;
b. classifying one or more objects based at least in part on or more factors; and
c. applying a distortion filter.

78. The method of claim 73, wherein the one or more parameters further comprise an age of the subject.

79. The method of claim 73, wherein the simulated field of view is generated in real-time.

80. The method of claim 73, wherein the altering further comprises blurring components of the simulated field of view.

81. The method of claim 73, wherein the one or more parameters further comprise a severity of the ophthalmic condition.

82. The method of claim 73, wherein the altering further comprises blurring text located less than or equal to a provided distance from a point of origin of the simulated field of view.

83. The method of claim 82, wherein the provided distance is adjusted dynamically in real-time.

84. The method of claim 73, wherein d) further comprises generating a simulated field of view affected by presbyopia, at least in part by altering objects located within 40 centimeters from the head-mounted display.

85. The method of claim 73, wherein d) further comprises generating a simulated field of view affected by a high level of presbyopia, at least in part by altering objects located within 90 centimeters from the head-mounted display.

86. A non-transitory computer-readable storage medium with instructions encoded thereon, the instructions executable by one or more processors to perform a method of simulating a field of view affected by an ophthalmic condition, the method comprising:
a) receiving, via from a user interface, a first input comprising the ophthalmic condition, one or more parameters relating to the ophthalmic condition, or a combination thereof;
b) acquiring, using a set of sensors of a head-mounted display, real-time sensor data comprising a video stream;
c) receiving, from a the head-mounted display, input the real-time sensor data and an input video stream;
d) using one or more computer processors, applying an algorithm processing the real-time sensor data to modify automatically alter at least a portion of the input video stream to generate a simulated field of view, wherein the modification is altering comprises generating a simulated field of view affected by the ophthalmic condition, based at least in part on the input real-time sensor data, the input video stream, and the one or more parameters; and
e) providing to a user, via a graphical interface, an electronic output comprising the simulated field of view affected by an the ophthalmic condition to a user via a graphical interface,
wherein the ophthalmic condition is selected from the group consisting of presbyopia, myopia, cataracts, astigmatism, macular degeneration, visual field loss due to glaucoma, and hemianopsia secondary to cerebral vascular accident or stroke.
